(19) United States
(12) Patent Application Publication
(10) Pub. No.: US 2026/0365598 A1
(43) Pub. Date: Nov. 19, 2026

(54) METHODS FOR FORMING LIGHT OLEFINS UTILIZING OXIDATION VESSELS

(71) Applicant: DOW GLOBAL TECHNOLOGIES LLC, Midland, MI (US)

(72) Inventors: Lin Luo, Sugarland, TX (US); Adrianus Koeken, Terneuzen (NL); Andrzej Malek, Midland, MI (US); Brian W. Goodfellow, Sugarland, TX (US); Matthew T. Pretz, Lake Jackson, TX (US); Liwei Li, Lake Jackson, TX (US); Quan Yuan, Lake Jackson, TX (US)

(73) Assignee: DOW GLOBAL TECHNOLOGIES LLC, Midland, MI (US)

(21) Appl. No.: 19/129,888

(22) PCT Filed: Nov. 22, 2023
(86) PCT No.: PCT/US2023/080911

§ 371 Date: May 14, 2025

Related U.S. Application Data
(60) Provisional application No. 63/428,500, filed on Nov. 29, 2022.

(51) Int. Cl. C07C 5/48; B01J 23/08; B01J 23/42

(52) U.S. Cl. CPC C07C 5/48; B01J 23/08; B01J 23/42; C07C 2523/08; C07C 2523/42

(57) ABSTRACT

Light olefins may be formed by a method that may include reacting a feed stream in the presence of a catalyst in a reactor to form a product stream and a deactivated catalyst including coke, separating at least a portion of the product stream from the deactivated catalyst, and passing the deactivated catalyst to an oxidation vessel and contacting the deactivated catalyst with a first oxygen-containing gas to remove at least a portion of the coke on the deactivated catalyst to produce a decoked catalyst. The method may further include passing the decoked catalyst to a combustor and combusting a supplemental fuel in the combustor to heat the decoked catalyst and produce a heated catalyst, passing the heated catalyst to an oxygen soak zone and contacting the heated catalyst with a second oxygen-containing gas to produce a reactivated catalyst, and passing the reactivated catalyst to the reactor.

### CROSS-REFERENCE TO RELATED APPLICATIONS

This application claims the benefit of U.S. Provisional Application Ser. No. 63/428,500 filed Nov. 29, 2022, the entire disclosure of which is hereby incorporated herein by reference.

### TECHNICAL FIELD

Embodiments described herein generally relate to chemical processing and, more specifically, to methods and systems for light olefin production.

### BACKGROUND

Light olefins, such as propylene, may be used as base materials to produce many different materials, such as polypropylene, isopropanol, and acrylic acid, which may be used in, e.g., packaging, construction, and textiles. As a result of this utility, there is a worldwide demand for light olefins. Suitable processes for producing light olefins generally depend on the given chemical feed and include those that utilize fluidized catalysts. For example, light olefins may be formed by the catalytic dehydrogenation of alkanes in a fluidized bed reactor. However, there is a need for improvement in the systems and associated catalysts used to make light olefins.

### SUMMARY

Some methods of producing light olefins utilize a catalyst that may become coked following dehydrogenation. In conventional embodiments, this coked catalyst may then be directly introduced into a combustor, where coke is removed from the catalyst by combustion of the coke with oxygen (for example, air) and where a supplemental fuel in the combustor is further utilized to heat the catalyst. The heated catalyst may then be sent to an air soak zone where catalyst is reactivated by an oxygen containing gas, such as air, prior to it being passed back to the dehydrogenation reaction reactor. It has been discovered, presently, that, in some embodiments, catalyst reactivation is not efficient by the conventional reactivation processes utilizing a single combustor for coke burning. For example, it has been discovered that insufficient amounts of coke may be removed, or insufficient structural change of catalyst active sites (due to limited contact with oxygen before catalyst interacts with fuel gas), or both, may be present in such comparative embodiments. Described herein are methods for forming light olefins that utilize oxidation vessels. The oxidation vessels may generally be upstream of the combustors. In some embodiments, some amount of coke may be combusted and/or some structural changes of catalyst active sites may occur in the presence of oxygen in the oxidation vessel before it reaches the combustor.

According to one or more embodiments of the present disclosure, light olefins may be formed by a method that may comprise reacting a feed stream in the presence of a catalyst in a reactor to form a product stream and a deactivated catalyst comprising coke, separating at least a portion of the product stream from the deactivated catalyst, and passing the deactivated catalyst to an oxidation vessel and contacting the deactivated catalyst with a first oxygen-containing gas to remove at least a portion of the coke on the deactivated catalyst to produce a decoked catalyst. The method may further comprise passing the decoked catalyst to a combustor and combusting a supplemental fuel in the combustor to heat the decoked catalyst and produce a heated catalyst, passing the heated catalyst to an oxygen soak zone and contacting the heated catalyst with a second oxygen-containing gas to produce a reactivated catalyst, and passing the reactivated catalyst to the reactor.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawing and claims, or recognized by practicing the described embodiments. The drawing is included to provide a further understanding of the embodiments and, together with the detailed description, serves to explain the principles and operations of the claimed subject matter. However, the embodiment depicted in the drawing is illustrative and exemplary in nature, and not intended to limit the claimed subject matter.

### BRIEF DESCRIPTION OF THE DRAWINGS

The following detailed description may be better understood when read in conjunction with the following drawings, in which:

FIG. 1 schematically depicts a reactor system, according to one or more embodiments of the present disclosure;

FIG. 2 schematically depicts another reactor system, according to one or more embodiments of the present disclosure;

FIG. 3 schematically depicts another reactor system, according to one or more embodiments of the present disclosure;

FIG. 4 schematically depicts another reactor system, according to one or more embodiments of the present disclosure;

FIG. 5 schematically depicts another reactor system, according to one or more embodiments of the present disclosure; and

FIG. 6 schematically depicts another reactor system, according to one or more embodiments of the present disclosure.

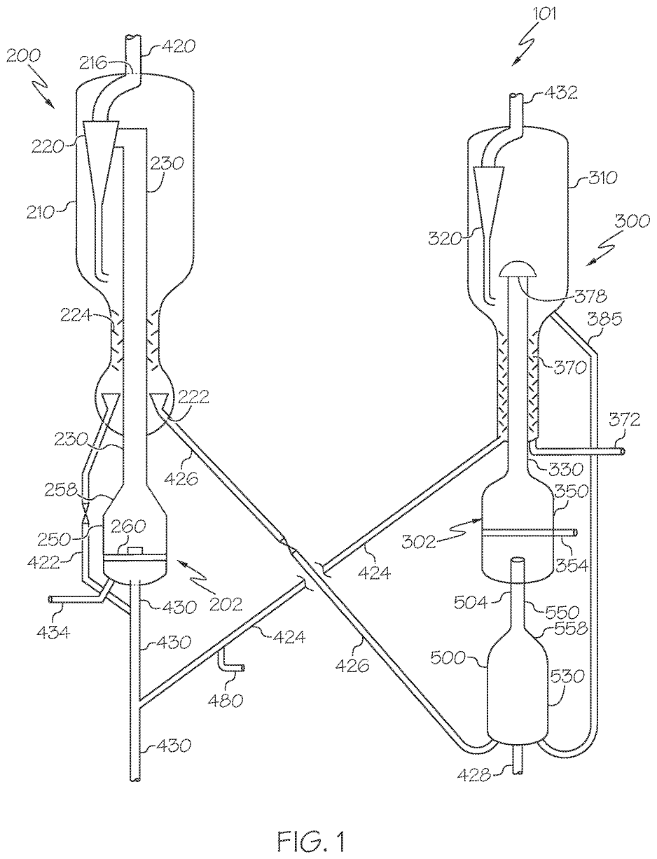

When describing the schematic illustrations of FIGS. 1-6, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Reference will now be made in greater detail to various embodiments, some of which are illustrated in the accompanying drawings.

### DETAILED DESCRIPTION

Embodiments presently disclosed are described in detail herein in the context of the reactor systems of FIGS. 1-6 operating as fluidized dehydrogenation reactor systems that produce light olefins. However, it should be understood that the principles disclosed and taught herein may be applicable to other systems which utilize different system components oriented in different ways, or different reaction schemes utilizing various catalyst compositions. For example, the concepts described may be equally applied to other systems with alternate reactor units and regeneration units, such as those that operate under non-fluidized conditions or include downers rather than risers. Additionally, it is contemplated that light olefins may be produced from a variety of hydrocarbon feed streams and by utilizing different reaction mechanisms. For example, light olefins may be catalytically produced by at least dehydrogenation reactions, cracking reactions, dehydration reactions, and methanol-to-olefin reactions. In some embodiments, oxygen carrier materials may also be utilized, as is described herein. These reaction types may utilize different feed streams and/or different catalysts to produce light olefins. It should be further understood that not all portions of FIGS. 1-6 should be construed as essential to the claimed subject matter.

Now referring to FIG. 1, an example reactor system 101 that may be suitable for use with the methods and/or apparatuses described herein is schematically depicted. The reactor system 101 generally comprises multiple system components, such as a reactor portion 200 and a catalyst processing portion 300. As described herein, “system components” refer to portions of the reactor system 101, such as reactors, separators, transfer lines, combinations thereof, and the like. As used herein in the context of FIG. 1, the reactor portion 200 generally refers to the portion of a reactor system 101 in which the major process reaction takes place (e.g., dehydrogenation) to form the product stream. A feed stream enters the reactor portion 200, is converted to a product stream (containing product and unreacted feed), and exits the reactor portion 200. The reactor portion 200 comprises a reactor 202 which may include an upstream reactor section 250 and a downstream reactor section 230. According to one or more embodiments, as depicted in FIG. 1, the reactor portion 200 may additionally include a catalyst separation section 210, which serves to separate the catalyst from the chemical products formed in the reactor 202. Also, as used herein, the catalyst processing portion 300 generally refers to the portion of the reactor system 101 where the catalyst is in some way processed, such as by combustion, to, e.g., improve catalytic activity by decoking and/or heating the catalyst. The catalyst processing portion 300 may comprise an oxidation vessel 500, a combustor 350, a riser 330, and may additionally comprise a catalyst separation section 310. In one or more embodiments, the catalyst separation section 210 of the reactor portion 200 may be in fluid communication with the oxidation vessel 500 (e.g., via standpipe 426) and the catalyst separation section 310 may be in fluid communication with the upstream reactor section 250 (e.g., via standpipe 424 and transport riser 430).

Generally as is described herein, catalyst is cycled between the reactor portion 200 and the catalyst processing portion 300. It should be understood that when “catalysts” are referred to herein, they may refer to solid materials that are catalytically active for a desired reaction, or may equally refer to other particulate solids referenced with respect to the system which do not necessarily have catalytic activity but affect the reaction, such as oxygen carriers. The terms “catalytic activity” and “catalyst activity” refer to the degree to which the catalyst is able to catalyze the reactions conducted in the reactor system 101. The catalyst that exits the reactor portion 200 may be deactivated catalyst. As used herein, “deactivated” may refer to a catalyst which has reduced catalytic activity or is cooler as compared to catalyst entering the reactor portion 200. However, deactivated catalyst may maintain some catalytic activity. Reduced catalytic activity may result from contamination with a substance such as coke. Reactivation (sometimes called “regeneration” herein) may remove the contaminant such as coke, raise the temperature of the catalyst, or both. In embodiments, deactivated catalyst may be reactivated by catalyst reactivation in the catalyst processing portion 300. The deactivated catalyst may be reactivated by, but not limited to, removing coke by contacting the deactivated catalyst with an oxygen-containing gas to produce a decoked catalyst, removing coke by combustion, recovering catalyst acidity, oxidizing the catalyst, other reactivation processes, or combinations thereof. In some embodiments, the catalyst may be heated during reactivation by combustion of a supplemental fuel, such as hydrogen, methane, ethane, propane, natural gas, or combinations thereof, to produce a heated catalyst. The heated catalyst may contact an oxygen-containing gas in the oxygen soak zone 370 of the catalyst processing portion 300 to produce a reactivated catalyst. The reactivated catalyst from the catalyst processing portion 300 may then be passed back to the reactor portion 200.

As described with respect to FIG. 1, the feed stream may enter feed inlet 434 into the reactor 202, and the product stream may exit the reactor system 101 via pipe 420. According to one or more embodiments, the reactor system 101 may be operated by feeding a chemical feed (e.g., in a feed stream) and a fluidized catalyst into the upstream reactor section 250. The chemical feed contacts the catalyst in the upstream reactor section 250, and each flow upwardly into and through the downstream reactor section 230 to produce a chemical product.

The reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430 which, in operation, may provide reactivated catalyst in a feed stream to the reactor portion 200. The reactivated catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250, where in such embodiments some of the catalyst is not passed through the catalyst processing portion 300. The catalyst can also be fed via standpipe 422 directly to the upstream reactor section 250 (not depicted in FIG. 1). This catalyst may be somewhat deactivated, but may still, in some embodiments, be suitable for reaction in the upstream reactor section 250, particularly when used in combination with reactivated catalyst.

Still referring to FIG. 1, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 250 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at transport velocity, where the gas and catalyst have about the same velocity in a dilute phase.

According to embodiments, the chemical product and the catalyst may be passed out of the downstream reactor section 230 to a separation device 220 in the catalyst separation section 210, where the catalyst is separated from the chemical product, which is transported out of the catalyst separation section 210. According to one or more embodiments, following separation from vapors in the separation device 220, the catalyst may generally move through the stripper 224 to the catalyst outlet port 222 where the catalyst is transferred out of the reactor portion 200 via standpipe 426 and into the catalyst processing portion 300.

According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the invention.

According to one or more embodiments, and referring to FIG. 1, the deactivated catalyst may pass from the reactor portion 200 to an oxidation vessel 500 via standpipe 426. A first oxygen-containing gas, such as air, may enter the oxidation vessel 500, for example, via pipe 428, and contact the deactivated catalyst. The first oxygen-containing gas may contact the deactivated catalyst in the oxidation vessel 500 for 0.1 minutes to 10 minutes. The fluidization regime of the deactivated catalyst in the oxidation vessel 500 may be a dense phase transport, a bubbling bed, a turbulent fluidized bed, or a fast fluidized bed fluidization regime. The oxidation vessel 500 may include an inlet port 504 that may be in fluid communication with the combustor 350, such that the oxidation vessel 500 is directly connected to the combustor 350 and the decoked catalyst is passed directly from the oxidation vessel 500 to the combustor 350. The deactivated catalyst contacting the first oxygen-containing gas for the duration of from 0.1 minutes to 10 minutes may remove at least a portion of the coke deposited on the deactivated catalyst to produce a decoked catalyst. It is contemplated that the “decoked” catalyst may still include some amount of coke, but less coke that catalyst entering oxidation vessel 500. The decoked catalyst may pass through the inlet port 504 of the oxidation vessel 500 and into the combustor 350. Without being bound by a theory, it is believed that contacting the deactivated catalyst with the first oxygen-containing gas in the oxidation vessel 500 will remove at least a portion of coke from the deactivated catalyst, which will prevent excessive oxygen depletion in the combustor 350 in the event of maldistribution of the catalyst in the combustor 350.

In one or more embodiments, the deactivated catalyst may comprise from 0.01 wt. % to 0.4 wt. % coke, based on the total weight of the catalyst, when entering the oxidation vessel 500 and after exiting the reactor portion 200 via standpipe 426. In additional embodiments, the deactivated catalyst may comprise from 0.01 wt. % to 0.1 wt. %, from 0.1 wt. % to 0.2 wt. %, from 0.2 wt. % to 0.3 wt. %, from 0.3 wt. % to 0.4 wt. %, or any combination of these ranges, of coke, based on the total weight of the catalyst, when entering the oxidation vessel 500 and after exiting the reactor portion 200 via standpipe 426.

Without being bound by any particular theory, it is believed that the coke present on the deactivated catalyst may include different types of coke, referred to as “hard” and “soft” coke herein, where hard coke may be more difficult to combust than soft coke. In one or more embodiments, the process in the oxidation vessel 500 may combust the hard coke, which if not removed, may decrease the catalytic regeneration the catalyst experiences in the combustor 350 and in the oxygen soak zone 370.

In one or more embodiments, at least 70 wt. % of coke is removed from the deactivated catalyst in the oxidation vessel 500 to produce the decoked catalyst. For example, in some embodiments, at least 75 wt. %, at least 80 wt. %, at least 85 wt. %, at least 90 wt. %, at least 95 wt. %, at least 99 wt. %, or even 100 wt. % of coke is removed from the deactivated catalyst in the oxidation vessel 500 to produce the decoked catalyst.

In one or more embodiments, from 0 wt. % to 30 wt. % of coke may be present in the decoked catalyst, such as that passed from the oxidation vessel 500 to the combustor 350. For example, from 0 wt. % to 5 wt. %, from 5 wt. % to 10 wt. %, from 10 wt. % to 15 wt. %, from 15 wt. % to 20 wt. %, from 20 wt. % to 25 wt. %, from 25 wt. % to 30 wt. %, or any combination of these ranges, of coke is present in the decoked catalyst, such as that passed from the oxidation vessel 500 to the combustor 350.

In one or more embodiments, at least 95 wt. % of the coke on the deactivated catalyst is combusted in the oxidation vessel. In additional embodiments, at least 96 wt. %, at least 97 wt. %, at least 98 wt. %, at least 99 wt. % or even 100 wt. % of the coke on the deactivated catalyst is combusted in the oxidation vessel.

As is depicted in FIG. 1, the oxidation vessel 500 may have a shape similar or identical to that described in the context of the reactor 202. For example, oxidation vessel 500 may include an upstream section 550, a transition section 558, and a downstream section 530. As depicted in FIG. 1, the upstream section 550 may be positioned below the downstream section 530. Such a configuration may be referred to as an upflow configuration in the oxidation vessel 500. The upstream section 550 may include a vessel, drum, barrel, vat, or other container suitable for a given chemical reaction. As depicted in FIG. 1, the upstream section 550 may be connected to the downstream section 530 via the transition section 558. The upstream section 550 may generally comprise a greater cross-sectional area than the downstream section 530. The transition section 558 may be tapered from the size of the cross-section of the upstream section 550 to the size of the cross-section of the downstream section 530 such that the transition section 558 projects inwardly from the upstream section 550 to the downstream section 530. For example, the transition section 558 may be a frustum.

Still referring to FIG. 1, in one or more embodiments, based on the shape, size, and other processing conditions (such as temperature and pressure) in the upstream section 530 and the downstream section 550, the upstream section 530 may operate as a fluidized bed, such as in a fast fluidized, turbulent, or bubbling bed upflow reactor, while the downstream section 550 may operate in more of a plug flow manner, such as in a riser reactor. For example, the oxidation vessel 500 of FIG. 1 may comprise an upstream section 530 operating as a fast fluidized, turbulent, or bubbling bed reactor and a downstream section 550 operating as a dilute phase riser reactor, with the result that the average catalyst and gas flow moves concurrently upward.

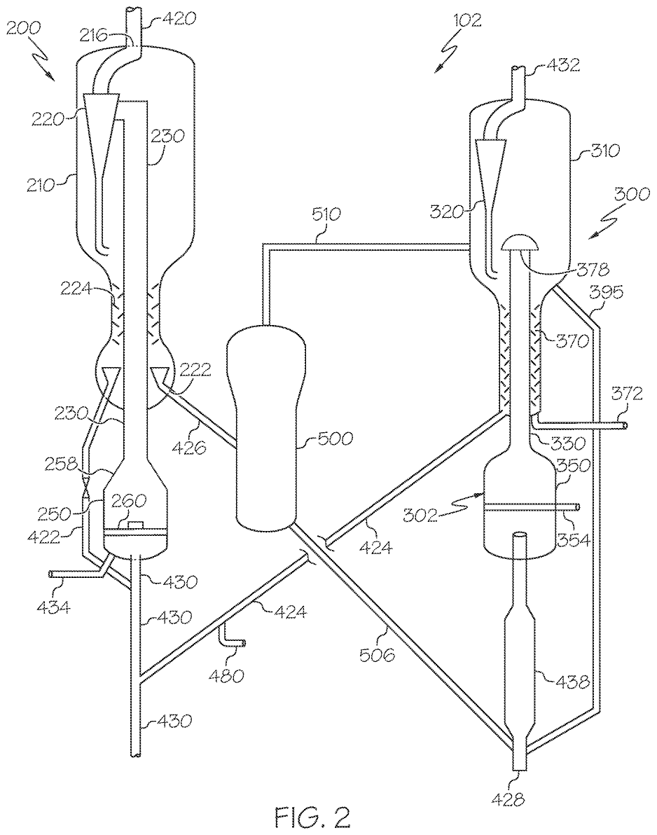

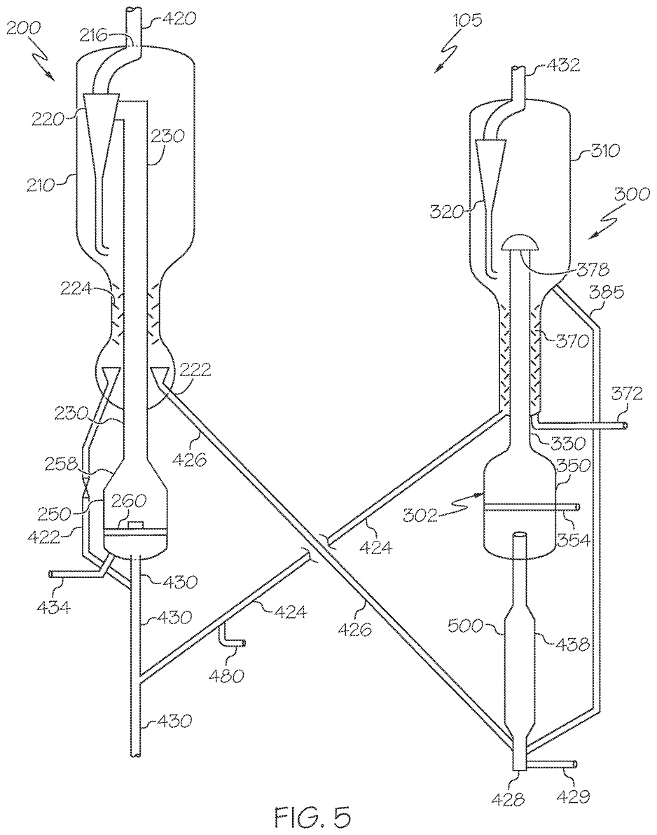

In one or more embodiments, the oxidation vessel 500 may operate with a superficial gas velocity of less than 8 ft/s in at least the upstream section 550. In one or more embodiments, the oxidation vessel 500 may operate with a superficial gas velocity of from 0.1 ft/s to 8 ft/s, such as from 0.1 ft/s to 1 ft/s, from 1 ft/s to 2 ft/s, from 2 ft/s to 3 ft/s, from 3 ft/s to 4 ft/s, from 4 ft/s to 5 ft/s, from 5 ft/s to 6 ft/s, from 6 ft/s to 7 ft/s, from 7 ft/s to 8 ft/s, or any combination of these ranges. In one or more embodiments, the decoked catalyst in the combustor 350 may have a catalyst bed density of from 20 lb/ft³ to 60 lb/ft³, such as from 20 lb/ft³ to 30 lb/ft³, from 30 lb/ft³ to 40 lb/ft³, from 40 lb/ft³ to 50 lb/ft³, from 50 lb/ft³ to 60 lb/ft³, or any combination of these ranges. The superficial gas velocity may substantially accelerate through the downstream section 550 of the oxidation vessel 500. In one or more embodiments, the oxidation vessel 500 may operate with a catalyst flux of from 100 to 300 lb/ft² sec, such as from 100 to 150 lb/ft² sec, from 150 to 200 lb/ft² sec, from 200 to 250 lb/ft² sec, from 250 to 300 lb/ft² sec, or any combination of these ranges. In embodiments such as that in FIG. 2 or 5 utilizing a pipe 438 as an oxidation vessel, these catalyst characteristics may also be present. The catalyst residence in this pipe configuration can range from 10 to 240 seconds, 20 to 120 seconds, or 25 to 60 seconds.

As is depicted in FIG. 1, the oxidation vessel 500 may be directly connected to the combustor 350 such that the catalyst is fluidized while in the oxidation vessel 500 and directly fed to the combustor 350.

Referring still to FIG. 1, in the combustor 350, the catalyst may be processed by, for example, combustion of oxygen with supplemental fuel. For example, and without limitation, the catalyst may be further decoked and/or supplemental fuel may be combusted to heat the catalyst. Combusting the supplemental fuel in the combustor 350 may increase the temperature of the decoked catalyst to greater than or equal to 660 °C. The catalyst may then be passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel, referred to herein as flue gas). The flue gas may pass out of the catalyst processing portion 300 via outlet pipe 432. The separated catalyst is then passed through the oxygen soak zone 370 within the catalyst separation section 310 to the upstream reactor section 250 via standpipe 424 and transport riser 430, where it is further utilized in a catalytic reaction. Thus, the catalyst, in operation, may cycle between the reactor portion 200 and the catalyst processing portion 300. In general, the processed chemical streams, including the feed streams and product streams may be gaseous, and the catalyst may be fluidized particulate solid.

As is described herein, since the majority of the coke is eliminated in the oxidation vessel 500, according to one or more embodiments, potential maldistribution of such catalyst in the combustor 350 may not lead to as extreme of local concentration differences in oxygen in the combustor 350.

Without being bound by any particular theory, it is believed that, in conventional embodiments, the deactivated catalyst that is coked may not be stoichiometrically distributed well with air when entering into the combustor and, subsequently, contacting fuel gas. It is believed that this lack of local oxygen may be the result of the catalyst being maldistributed in the combustor. Such maldistribution may result in poor coke removal from catalyst and, consequently, deficiency in activity to combust the supplemental fuel. As such, in some embodiments described herein, the above-described effects of catalyst maldistribution in the combustor can be minimized since less coke is available to locally decrease oxygen concentration in the combustor.

Additionally, and without being bound by any theory, it is believed that in some embodiments, the maldistribution of catalyst with coke can result in areas of the combustor with deficient concentrations of oxygen due to the localized combustion of the coke (reducing local oxygen concentrations in those areas). As such, these areas may have insufficient oxygen to combust the supplemental fuel at the desired rate and system inefficiencies maybe, therefore, present. Without being bound by any particular theory, it is believed that such catalyst (by conventional processes) requires longer reactivation to reach the same dehydrogenation activity. The embodiments described herein may, therefore, improve catalytic performance and overall system efficiency and product yield generation.

In additional embodiments, and without being bound by theory, it is believed that there may be be other mechanisms in conventional process (e.g., those without oxidation vessels as described herein), such as lack of needed structural change for catalyst active sites, when the contact with oxygen is limited before catalyst contacts with fuel gas. As such, in some embodiments, the utilization of the oxidation vessel may improve structural change in the catalyst that is sometimes desirable when contacted with fuel gas.

Referring now to the catalyst processing portion 300, as depicted in FIG. 1, the combustor 350 of the catalyst processing portion 300 may be in fluid communication with the riser 330. Oxygen-containing gas, such as air, may be passed via pipe 428 and through the oxidation vessel 500, or a separate oxygen-containing gas line may pass directly into the combustor 350 (not depicted). The combustor 350 and riser 330, collectively referred to as the catalyst combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream, to the combustor 350.

In one or more embodiments, the combustor 350 may operation with a superficial gas velocity of less than 4 ft/s. In one or more embodiments, the combustor 350 may operate with a superficial gas velocity of from 0.1 ft/s to 4 ft/s, such as from 0.1 ft/s to 3.5 ft/s, from 0.1 ft/s to 3 ft/s, from 0.1 ft/s to 2.5 ft/s, from 0.1 ft/s to 2 ft/s, from 0.1 ft/s to 1.5 ft/s, from 0.1 ft/s to 1 ft/s, from 0.5 ft/s to 4 ft/s, from 1 ft/s to 4 ft/s, from 1.5 ft/s to 4 ft/s, from 2 ft/s to 4 ft/s, from 2.5 ft/s to 4 ft/s, from 3 ft/s to 4 ft/s, from 0.5 ft/s to 3.5 ft/s, from 1 ft/s to 3 ft/s, or from 1.5 ft/s to 2.5 ft/s. In one or more embodiments, the decoked catalyst in the combustor 350 may have a catalyst bed density of from 20 lb/ft³ to 40 lb/ft³, such as from 25 lb/ft³ to 35 lb/ft³, from 30 lb/ft³ to 35 lb/ft³, from 20 lb/ft³ to 30 lb/ft³, or from 20 lb/ft³ to 25 lb/ft³.

As described in one or more embodiments, following separation of flue gas from catalyst in the riser termination separator 378 and secondary separation device 320, treatment of the processed catalyst with the second oxygen-containing gas is conducted in the oxygen soak zone 370. In some embodiments, the oxygen soak zone 370 includes a fluid solids contacting device. The fluid solids contacting device may include baffles or grid structures to facilitate contact of the processed catalyst with the second oxygen-containing gas. Examples of fluid solid contacting devices are described in further detail in U.S. Pat. Nos. 9,827,543 and 9,815,040. The fluidization regime within the oxygen soak zone 370 may be bubbling bed type fluidization. The oxygen soak zone 370 may include an oxygen-containing gas inlet 372, which may supply the second oxygen-containing gas to the oxygen soak zone 370 for oxygen treatment of the catalyst. The temperature of the heated catalyst may be greater than or equal to 660 °C while the heated catalyst is in the oxygen soak zone 370. Without being bound by a theory, it is believed that contacting the heated catalyst with an oxygen-containing gas in the oxygen soak zone 370 increases the catalyst activity of dehydrogenating an alkane and produces an increased alkane conversion in the reactor portion 200.

In one or more embodiments, the reactivated catalyst that is produced from treating the heated catalyst with the second oxygen-containing gas in the oxygen soak zone 370 may further be contacted with a stripping gas before passing the reactivated catalyst to the reactor portion 200. In some embodiments, the stripping gas may be nitrogen, methane or steam or one or more inert gases. Without being bound by a theory, it is believed that contacting the reactivated catalyst with a stripping gas removes at least a portion of molecular oxygen trapped within or between catalyst particles that will reduce the amount of oxygen carried over to reactor 200.

As is depicted in FIG. 1, in one or more embodiments, a portion of the heated catalyst may exit the catalyst processing portion 300 before passing to the oxygen soak zone 370. In some embodiments, a recycled portion of the heated catalyst from the catalyst processing portion 300 may pass directly to the oxidation vessel 500 via line 385. Such recycled catalyst may be exposed to some oxygen, but not the normal amount of oxygen associated with the oxygen soak zone 370.

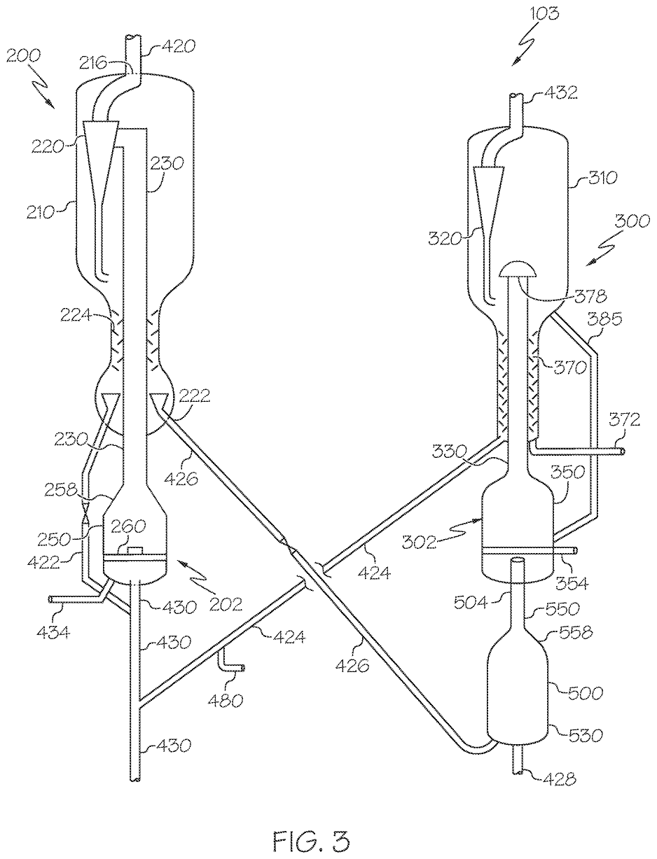

Now referring to FIG. 3, another embodiment of a reactor system 103 is depicted which is similar or identical to that of FIG. 1 aside from the differences described hereinbelow. In the embodiment of FIG. 3, recycled catalyst in line 385 is passed to the combustor 350 rather than to the oxidation vessel 500 (as described in the embodiment of FIG. 1). Such a scheme may be effective since the recycled catalyst is largely void of coke, so maldistribution of the catalyst in the combustor 350 is not as prone to cause problems.

Another embodiment is depicted in FIG. 2 illustrating a reactor system 102, which is similar or identical to FIG. 1 which is similar or identical to that of FIG. 1 aside from the differences described hereinbelow. In the embodiment of FIG. 2, the oxidation vessel 500 and the combustor 350 may be physically isolated from one another. In such embodiments, the oxidation vessel 500 and the combustor 350 are connected via one or more pipelines and the catalyst may not be exposed to oxygen and/or may not be fluidized throughout the entirety of such pipelines. As depicted in FIG. 2, the oxidation vessel 500 may be a separate reaction vessel that operates as a fluidized bed. Oxygen-containing gas (inlet not shown in FIG. 2) may contact the deactivated catalyst entering the oxidation vessel 500 via line 426. Line 506 may pass the catalyst, now decoked, to pipe 438, where oxygen-containing gas via 428 fluidizes the catalyst and passes the catalyst through pipe 438 into the combustor 350. Some additional coke combustion may occur in pipe 438, but the majority of coke combustion may occur in oxidation vessel 500. Line 510 may pass the gas (after treatment) from oxidation vessel 500 to the catalyst separation section 310 where the gas and small quantity of catalyst carry-over can be separated using the existing cyclones in the catalyst processing system 300, as is shown in FIG. 2.

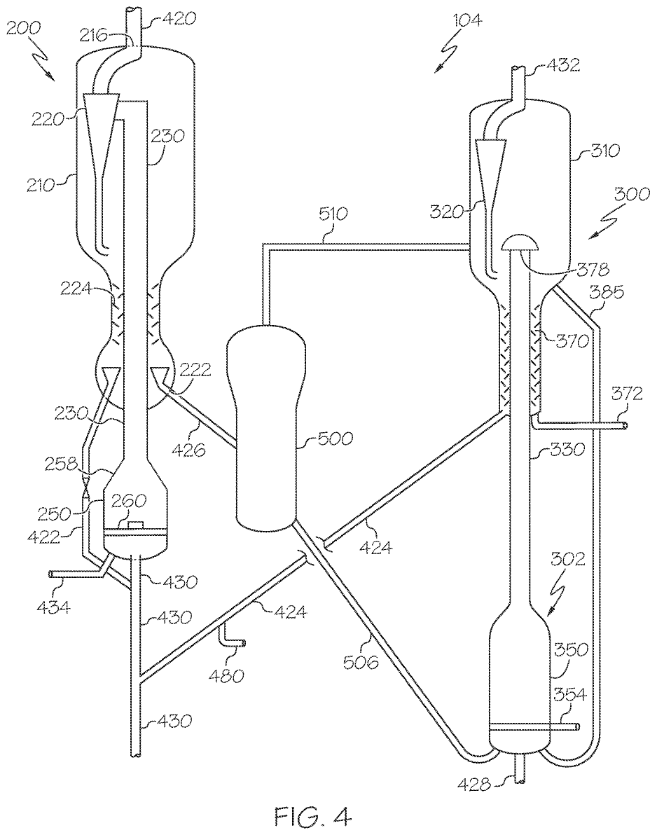

Another embodiment is depicted in FIG. 4 illustrating reactor system 104, which is similar or identical in many respects to the embodiment of FIG. 2. In the embodiment of FIG. 4, the isolated oxidation vessel 500 (as described in the context of FIG. 4, previously) de-cokes the catalyst. However, rather than passing the catalyst via line 506 to a pipe 438 where oxygen-containing gas fluidizes the catalyst, the catalyst is passed via line 506 directly into the combustor 350. In such embodiments, it is believed that little or no coke is burned in the passage between the oxidation vessel 500 and the combustor 350, as compared with the embodiment of FIG. 2 where combustion may occur, in addition to in the oxidation vessel 500, in the pipe 438.

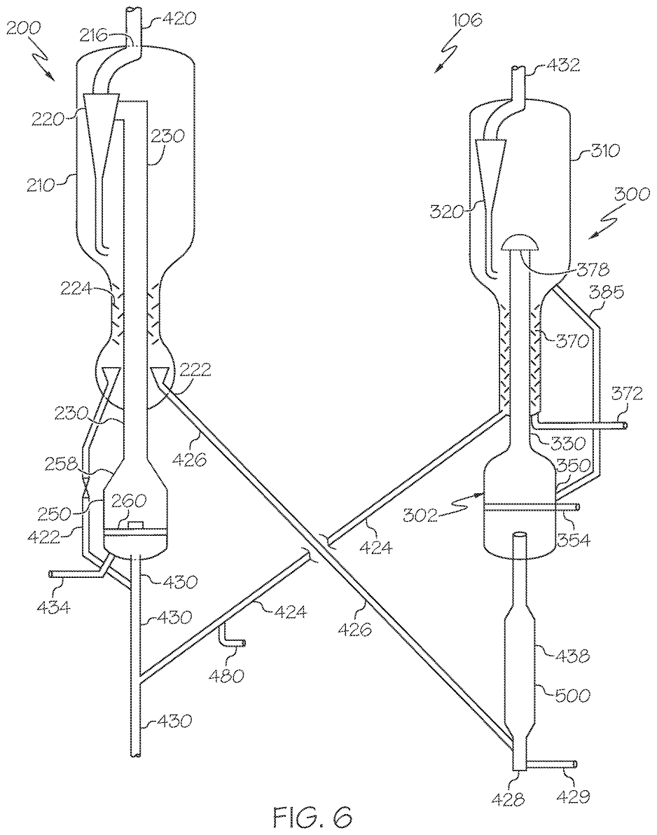

Now referring to the reactor system 105 of FIG. 5, another embodiment is depicted, which may be similar or identical in many respects to FIG. 1. In FIG. 5, the oxidation vessel 500 is a pipe 438 where oxygen-containing gas from port 428 via line 429 passed into the pipe 438 and fluidizes the catalyst to pass it upwards into the combustor 350. In such embodiments, a separate reaction drum is not included. Rather, the oxidation vessel 500 is a pipe, which may have dilute phase fluidization. FIG. 5 depicts an embodiment where catalyst is recycled via line 385 into the oxidation vessel 50, similar to the embodiment of FIG. 1. FIG. 6 depicts yet another embodiment, reactor system 106, which is similar or identical to that of FIG. 5 but recycles catalyst via line 385 directly to the combustor 350 rather than to the oxidation vessel 500.

In one or more embodiments, the light olefins may be present in a “product stream” sometimes called an “olefin-containing effluent” and include light olefins. Such a stream exits the reactor system 102 and may be subsequently processed. As used in the present disclosure, the term “light olefins” refers to one or more of styrene, ethylene, propylene, and butene. The term butene includes any isomers of butene, such as α-butylene, cis-β-butylene, trans-β-butylene, and isobutylene. In some embodiments, the olefin-containing effluent includes at least 25 wt. % light olefins based on the total weight of the olefin-containing effluent. For example, the olefin-containing effluent may include at least 35 wt. % light olefins, at least 45 wt. % light olefins, at least 55 wt. % light olefins, at least 65 wt. % light olefins, or at least 75 wt. % light olefins based on the total weight of the olefin-containing effluent. The olefin-containing effluent may further comprise unreacted components of the feed stream, as well as other reaction products that are not considered light olefins. The light olefins may be separated from unreacted components in subsequent separation steps.

In non-limiting examples, the reactor systems described herein may be utilized to produce light olefins from hydrocarbon feed streams. Light olefins may be produced from a variety of hydrocarbon feed streams by utilizing different reaction mechanisms. For example, light olefins may be produced by at least dehydrogenation reactions, cracking reactions, dehydration reactions, and methanol-to-olefin reactions. These reaction types may utilize different feed streams and different particulate solids to produce light olefins. It should be understood that when “catalysts” are referred to herein, they may equally refer to the particulate solid referenced with respect to the system of FIG. 1.

According to one or more embodiments, the reaction may be a dehydrogenation reaction. According to such embodiments, the hydrocarbon feed stream may comprise one or more of ethyl benzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethyl benzene. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

In one or more embodiments, the dehydrogenation reaction may utilize gallium and/or platinum particulate solids as a catalyst. In such embodiments, the particulate solids may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction.

In one or more embodiments, the reaction mechanism may be dehydrogenation followed by combustion (in the same chamber). In such embodiments, a dehydrogenation reaction may produce hydrogen as a byproduct, and an oxygen carrier material may contact the hydrogen and promote combustion of the hydrogen, forming water. Examples of such reaction mechanisms, which are contemplated as possible reactions mechanisms for the systems and methods described herein, are disclosed in WO 2020/046978, the teachings of which are incorporated by reference in their entirety herein.

According to one or more embodiments, the reaction may be a cracking reaction. According to such embodiments, the hydrocarbon feed stream may comprise one or more of naphtha, n-butane, or i-butane. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of naphtha. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of naphtha, n-butane, and i-butane.

In one or more embodiments, the cracking reaction may utilize one or more zeolites as a catalyst. In such embodiments, the particulate solids may comprise one or more zeolites. In some embodiments, the one or more zeolites utilized in the cracking reaction may comprise a ZSM-5 zeolite. However, it should be understood that other suitable catalysts may be utilized to perform the cracking reaction. For example, suitable catalysts that are commercially available may include Intercat Super Z Excel or Intercat Super Z Exceed. In additional embodiments, the cracking catalyst may comprise, in addition to a catalytically active material, platinum. For example, the cracking catalyst may include from 0.001 wt. % to 0.05 wt. % of platinum. The platinum may be sprayed on as platinum nitrate and calcined at an elevated temperature, such as around 700 °C. Without being bound by theory, it is believed that the addition of platinum to the catalyst may allow for easier combustion of supplemental fuels, such as methane.

According to one or more embodiments, the reaction may be a dehydration reaction. According to such embodiments, the hydrocarbon feed stream may comprise one or more of ethanol, propanol, or butanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of butanol. In additional embodiments, the hydrocarbon feed stream or may comprise at least 50 wt. %, at least 60 wt. %, at least 70 wt. %, at least 80 wt. %, at least 90 wt. %, at least 95 wt. % or even at least 99 wt. % of the sum of ethanol, propanol, and butanol.

In one or more embodiments, the dehydration reaction may utilize one or more acid catalysts. In such embodiments, the particulate solids may comprise one or more acid catalysts. In some embodiments, the one or more acid catalysts utilized in the dehydration reaction may comprise a zeolite (such as ZSM-5 zeolite), alumina, amorphous aluminosilicate, acid clay, or combinations thereof. For example, commercially available alumina catalysts which may be suitable, according to one or more embodiments, include SynDol (available from Scientific Design Company), V200 (available from UOP), or P200 (available from Sasol). Commercially available zeolite catalysts which may be suitable include CBV 8014, CBV 28014 (each available from Zeolyst). Commercially available amorphous aluminosilicate catalysts which may be suitable include silica-alumina catalyst support, grade 135 (available from Sigma Aldrich). However, it should be understood that other suitable catalysts may be utilized to perform the dehydration reaction.

According to one or more embodiments, the reaction may be a methanol-to-olefin reaction. According to such embodiments, the hydrocarbon feed stream may comprise methanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of methanol.

In one or more embodiments, the methanol-to-olefin reaction may utilize one or more zeolites as a catalyst. In such embodiments, the particulate solids may comprise one or more zeolites. In some embodiments, the one or more zeolites utilized in the methanol-to-olefin reaction may comprise a one or more of a ZSM-5 zeolite or a SAPO-34 zeolite. However, it should be understood that other suitable catalysts may be utilized to perform the methanol-to-olefin reaction.

### EXAMPLES

Examples are provided herein which may disclose one or more embodiments of the present disclosure. However, the Examples should not be viewed as limiting on the claimed embodiments hereinafter provided.

### Example 1

Catalysts used in these experiments were made by a conventional incipient wetness method and contain platinum and gallium on alumina. The fresh catalyst has 300 ppm platinum, while the aged catalyst is a catalyst retrieved from pilot scale operation after nine month on stream and has 75 ppm platinum. Both catalysts have 1.6 wt. % gallium.

The experiments were carried out in a fixed bed lab-scale unit with simulated dehydrogenation, pre-combustion contact with air, combustion, and air soak steps. The catalyst was diluted with silicon carbide, 99.8 wt % purity 220 mesh Go Products, with a dilution of 1:2 by weight. The catalyst bed was held in place in the reactor with an upstream and downstream section of 20-40 mesh silicon carbide, 98.5 wt % purity 20-40 mesh Go Products. A cycle is built up as follows: pre-combustion treatment where the first gas stream is air with a weight hour space velocity of 7.1 hr⁻¹, where temperature and length of time the catalyst contacting the first gas stream are provided in Tables 1-3; combustion where the fuel gas stream is a feed of 2.5 vol. % methane/97.5 vol % synthetic air with a weight hour space velocity of 7.0 hr⁻¹, where the fuel gas stream was contacted with the catalyst for 3 minutes at 730 °C; air soak where the second gas stream is air with a weight hour space velocity of 7.1 hr⁻¹, where the second gas stream was contacted with the catalyst at 730 °C for a length of time provided in Tables 1-3; and dehydrogenation where the hydrocarbon stream is a feed of 90 mol. % propane/10 mol. % N₂ with a weight hour space velocity of 10 hr⁻¹ referred to propane, where the hydrocarbon stream was contacted with the catalyst at 625 °C for 1 minute. The dehydrogenation performance data were collected at 25 seconds time on stream and the combustion data were collected at 75 seconds time on stream.

Intermediate purge steps with inert gas were carried out to establish well-defined start and stop of the air soaks, combustion, and dehydrogenation steps. Generally, a 3 or 4 minute N₂ purge step (at about 7 hr⁻¹) was included between steps where there was no change in temperature. For steps with temperature change, the catalyst was heated up/cooled down under N₂ (at about 7 hr⁻¹) until the temperature was stabilized before introduction of the gas stream for the next step. Tables 1-3 below illustrate the propane conversion, propylene selectivity, and methane conversion achieved by following these experimental procedures and changing the temperature and reaction times of certain steps of the procedure. As can be seen, a higher propane conversion and propylene selectivity was achieved when the pre-combustion step was utilized and lower temperatures of the pre-combustion step were shown to achieve higher propane conversion and propylene selectivity.

TABLE 1
Fresh catalyst with the same 3 minute combustion followed by 7 or 10 minute
air soak, both at 730 °C. Data reported were results for Cycle 80.

| | Pre- | Combustion | Air Soak | Propane | Propylene | Methane |
|---|---|---|---|---|---|---|
| | Combustion | at 730 °C | at 730 °C | Conversion | Selectivity | Conversion |
| Description | (minutes) | (minutes) | (minutes) | (%) | (%) | (%) |
| No Pre- | 0 | 3 | 10 | 32.2 | 99.1 | 71.8 |
| Combustion | | | | | | |
| Pre- | 1 | 3 | 10 | 33.6 | 99 | 71.1 |
| Combustion | | | | | | |
| at 730 °C | | | | | | |
| Pre- | 5 | 3 | 10 | 34.1 | 99.1 | 70.8 |
| Combustion | | | | | | |
| at 730 °C | | | | | | |
| Pre- | 3 | 3 | 7 | 36.7 | 99.2 | 71.6 |
| Combustion | | | | | | |
| at 625 °C | | | | | | |

TABLE 2
Fresh catalyst with the same 3 minute combustion followed by 2 minute
air soak, both at 730 °C. Data reported were results for Cycle 80.

| | Pre- | Combustion | Air Soak | Propane | Propylene | Methane |
|---|---|---|---|---|---|---|
| | Combustion | at 730 °C | at 730 °C | Conversion | Selectivity | Conversion |
| Description | (minutes) | (minutes) | (minutes) | (%) | (%) | (%) |
| No Pre- | 0 | 3 | 2 | 24.6 | 98.7 | 72.6 |
| Combustion | | | | | | |
| Pre- | 5 | 3 | 2 | 28 | 99 | 69.8 |
| Combustion | | | | | | |
| at 730 °C | | | | | | |

TABLE 3
Aged catalyst with the same 3 minute combustion followed by 10 minute
air soak, both at 730 °C. Data reported were results for Cycle 150.

| | Pre- | Combustion | Air Soak | Propane | Propylene | Methane |
|---|---|---|---|---|---|---|
| | Combustion | at 730 °C | at 730 °C | Conversion | Selectivity | Conversion |
| Description | (minutes) | (minutes) | (minutes) | (%) | (%) | (%) |
| No Pre- | 0 | 3 | 10 | 32.5 | 99.1 | 58.9 |
| Combustion | | | | | | |
| Pre- | 3 | 3 | 10 | 33.7 | 99.2 | 56.5 |
| Combustion | | | | | | |
| at 625 °C | | | | | | |

### Example 2

Propane dehydrogenation performance after each step in the process was studied. 5 cases were run for a total of 79 cycles where the dehydrogenation step was conducted at 625 °C. for 1 minute, the pre-combustion air treatment step was conducted at 625 °C for 3 minutes, the combustion step was conducted at 730 °C for 3 minutes with a 2.5 vol. % methane/97.5 vol % synthetic air, and the air soak step was conducted at 730 °C for 7 minutes. Per case cycle 80 was varied to determine dehydrogenation performance after each step. In the first set with Pre-Combustion Air Treatment, Pre-combustion Air Treatment was conducted at 625 °C for 3 minutes, the combustion step was conducted at 730 °C for 3 minutes with 2.5 vol. % methane/97.5 vol % synthetic air, and the air soak step was conducted at 730 °C for 7 minutes. In the second set Pre-combustion Air Treatment was excluded, the combustion step was conducted at 730 °C for 3 minutes with 2.5 vol. % methane/97.5 vol % synthetic air, and the air soak step was conducted at 730 °C for 7 minutes. The dehydrogenation performance data were collected at 25 seconds time on stream. Table 4 illustrates the results of these experiments. As can be seen, the propane conversion percentage was higher after each step for the cycles that utilized the initial pre-combustion step.

TABLE 4
Comparison of dehydrogenation performance after each step in cycle
80, with and without the use of pre-combustion treatment

Propane Conversion (%)

| | With Pre- | Without Pre- |
|---|---|---|
| | Combustion | Combustion |
| | Air Treatment | Air Treatment |
| After Pre-Combustion | 31.2 | N/A |
| Treatment | | |
| After Combustion | 19.9 | 18.8 |
| After Air Soak | 36.7 | 33.1 |

The present disclosure includes several aspects. A first aspect is a method for forming light olefins, the method comprising: reacting a feed stream in the presence of a catalyst in a reactor to form a product stream and a deactivated catalyst comprising coke; separating at least a portion of the product stream from the deactivated catalyst; passing the deactivated catalyst to an oxidation vessel and contacting the deactivated catalyst with a first oxygen-containing gas to remove at least a portion of the coke on the deactivated catalyst to produce a decoked catalyst; passing the decoked catalyst to a combustor and combusting a supplemental fuel in the combustor to heat the decoked catalyst and produce a heated catalyst; passing the heated catalyst to an oxygen soak zone and contacting the heated catalyst with a second oxygen-containing gas to produce a reactivated catalyst; and passing the reactivated catalyst to the reactor.

Another aspect is any previous aspect or combination of previous aspects, wherein the deactivated catalyst comprises from 0.01 wt. % to 0.4 wt. % coke when entering the oxidation vessel.

Another aspect is any previous aspect or combination of previous aspects, wherein from 0 wt. % to 30 wt. % of coke is present in the decoked catalyst.

Another aspect is any previous aspect or combination of previous aspects, wherein at least 70 wt. % of the coke on the deactivated catalyst is combusted in the oxidation vessel.

Another aspect is any previous aspect or combination of previous aspects, wherein the first oxygen-containing gas is air.

Another aspect is any previous aspect or combination of previous aspects, wherein the oxidation vessel operates with a superficial gas velocity of less than 8 ft/s and the deactivated catalyst in the oxidation vessel has a catalyst bed density of from 20 lb/ft³ to 60 lb/ft³.

Another aspect is any previous aspect or combination of previous aspects, wherein the combustor operates with a superficial gas velocity of less than 4 ft/s when combusting the supplemental fuel and the decoked catalyst in the combustor has a catalyst bed density of from 20 lb/ft³ to 40 lb/ft³.

Another aspect is any previous aspect or combination of previous aspects, wherein combusting the supplemental fuel in the combustor increases the temperature of the decoked catalyst to greater than or equal to 660 °C.

Another aspect is any previous aspect or combination of previous aspects, wherein the temperature of the heated catalyst is greater than or equal to 660 °C while the heated catalyst is in the oxygen soak zone.

Another aspect is any previous aspect or combination of previous aspects, further comprising contacting the reactivated catalyst with a stripping gas before passing the reactivated catalyst to the reactor.

Another aspect is any previous aspect or combination of previous aspects, wherein the oxidation vessel is a fluidized bed reactor.

Another aspect is any previous aspect or combination of previous aspects, wherein the oxidation vessel is a fluidized pipe.

Another aspect is any previous aspect or combination of previous aspects, wherein the oxidation vessel is physically isolated from the combustor.

Another aspect is any previous aspect or combination of previous aspects, further comprising passing a recycled portion of the heated catalyst directly to the oxidation vessel.

Another aspect is any previous aspect or combination of previous aspects, further comprising passing a recycled portion of the heated catalyst directly to the combustor.

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” a second component, it is contemplated that, in some embodiments, the first component “consists” or “consists essentially of” that second component. It should further be understood that where a first component is described as “comprising” a second component, it is contemplated that, in some embodiments, the first component comprises at least 10%, at least 20%, at least 30%, at least 40%, at least 50%, at least 60%, at least 70%, at least 80%, at least 90%, at least 95%, or even at least 99% that second component (where % can be weight % or molar %).

For the purposes of describing and defining the present inventive technology, it is noted that reference herein to a variable being a “function” of a parameter or another variable is not intended to denote that the variable is exclusively a function of the listed parameter or variable. Rather, reference herein to a variable that is a “function” of a listed parameter is intended to be open ended such that the variable may be a function of a single parameter or a plurality of parameters.

It is also noted that recitations herein of “at least one” component, element, etc., should not be used to create an inference that the alternative use of the articles “a” or “an” should be limited to a single component, element, etc.

It is noted that recitations herein of a component of the present disclosure being “configured” in a particular way, to embody a particular property, or function in a particular manner, are structural recitations, as opposed to recitations of intended use. More specifically, the references herein to the manner in which a component is “configured” denotes an existing physical condition of the component and, as such, is to be taken as a definite recitation of the structural characteristics of the component.

As would be understood in the context of the term as used herein, the term “passing” may include directly passing a substance between two portions of the disclosed system and, in some other instances, to mean indirectly passing a substance between two portions of the disclosed system. For example, indirect passing may include steps where the named substance passes through an intermediate separation device, valve, sensor, etc.

For the purposes of describing and defining the present inventive technology it is noted that the terms “substantially” and “about” are utilized herein to represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

1. A method for forming light olefins, the method comprising:
reacting a feed stream in the presence of a catalyst in a reactor to form a product stream and a deactivated catalyst comprising coke;
separating at least a portion of the product stream from the deactivated catalyst;
passing the deactivated catalyst to an oxidation vessel and contacting the deactivated catalyst with a first oxygen-containing gas to remove at least a portion of the coke on the deactivated catalyst to produce a decoked catalyst;
passing the decoked catalyst to a combustor and combusting a supplemental fuel in the combustor to heat the decoked catalyst and produce a heated catalyst;
passing the heated catalyst to an oxygen soak zone and contacting the heated catalyst with a second oxygen-containing gas to produce a reactivated catalyst; and
passing the reactivated catalyst to the reactor.

2. The method of claim 1, wherein the deactivated catalyst comprises from 0.01 wt. % to 0.4 wt. % coke when entering the oxidation vessel.

3. The method of claim 1, wherein from 0 wt. % to 30 wt. % of coke is present in the decoked catalyst.

4. The method of claim 1, wherein at least 70 wt. % of the coke on the deactivated catalyst is combusted in the oxidation vessel.

5. The method of claim 1, wherein the first oxygen-containing gas is air.

6. The method of claim 1, wherein the oxidation vessel operates with a superficial gas velocity of less than 8 ft/s and the deactivated catalyst in the oxidation vessel has a catalyst bed density of from 20 lb/ft³ to 60 lb/ft³.

7. The method of claim 1, wherein the combustor operates with a superficial gas velocity of less than 4 ft/s when combusting the supplemental fuel and the decoked catalyst in the combustor has a catalyst bed density of from 20 lb/ft³ to 40 lb/ft³.

8. The method of claim 1, wherein combusting the supplemental fuel in the combustor increases the temperature of the decoked catalyst to greater than or equal to 660 °C.

9. The method of claim 1, wherein the temperature of the heated catalyst is greater than or equal to 660 °C while the heated catalyst is in the oxygen soak zone.

10. The method of claim 1, further comprising contacting the reactivated catalyst with a stripping gas before passing the reactivated catalyst to the reactor.

11. The method of claim 1, wherein the oxidation vessel is a fluidized bed reactor.

12. The method of claim 1, wherein the oxidation vessel is a fluidized pipe.

13. The method of claim 1, wherein the oxidation vessel is physically isolated from the combustor.

14. The method of claim 1, further comprising passing a recycled portion of the heated catalyst directly to the oxidation vessel.

15. The method of claim 1, further comprising passing a recycled portion of the heated catalyst directly to the combustor.
